(19) United States
(12) Patent Application Publication
(10) Pub. No.: US 2024/0264077 A1
(43) Pub. Date: Aug. 8, 2024

(54) GAS SENSOR DEVICE AND METHOD FOR UPDATING BASELINE CALIBRATION PARAMETER

(71) Applicant: Senseair AB, Delsbo (SE)

(72) Inventors: Cheng Yang, Stockholm (SE); Tobias Oechtering, Solna (SE); You Yang, Solna (SE)

(21) Appl. No.: 18/567,571

(22) PCT Filed: Jun. 10, 2022
(86) PCT No.: PCT/SE2022/050574

§ 371 Date: Dec. 6, 2023

(30) Foreign Application Priority Data
Jun. 14, 2021 (SE) 2150764-5

(51) Int. Cl. G01N 21/3504; G01N 21/27

(52) U.S. Cl. CPC G01N 21/3504; G01N 21/274; G01N 2201/12784

(57) ABSTRACT

A computer implemented method and a gas sensor device comprising a spectroscopic sensing unit (2), a memory (3) and a control unit (4), is described. The control unit (4) is configured to output calibrated values, which are measures of a concentration of a gas component measured by the spectroscopic sensing unit (2), wherein the calibrated values are determined from measurement values obtained from the spectroscopic sensing unit (2) and a baseline calibration parameter retrieved from the memory (3). The control unit is configured to update the baseline calibration parameter (zero) by identifying the minimum measurement value obtained during a predetermined first time period (14), obtaining a time for the first time period (14), obtaining a model value corresponding to the obtained time, determining an updated baseline calibration parameter based on the minimum measurement value and the model value, and updating the baseline calibration parameter stored in the memory (3).

### TECHNICAL FIELD

The present invention relates to a gas sensor device comprising a spectroscopic sensing unit such as an, e.g., non-dispersive infrared, NDIR, sensing unit. The gas sensor device is configured to output calibrated values, which are measures of a concentration of a gas component measured by the spectroscopic sensing unit, and are determined from measurement values obtained from the spectroscopic sensing unit and a baseline calibration parameter retrieved from the memory. The present invention also relates to a method for updating the baseline calibration parameter.

### BACKGROUND ART

Gas sensors are devices used to measure the presence or concentration of gases in an area and play an important role in many applications. Spectroscopic sensors are widely used for gas sensors, which rely on the Beer-Lambert law. A non-dispersive infrared, NDIR, sensor is a commonly used type of a spectroscopic sensor in which a nondispersive element is used to filter out the broadband light into a narrow spectrum suitable to sense a specific gas. However, spectroscopic sensors have been recognized to be sensitive to variations of ambient temperature, atmospheric pressure, humidity and some other environmental factors. Moreover, aging of the sensor components also results in inaccuracy of the sensors. Due to this, regular calibration is needed for long-term accuracy of the sensors.

Today, state of the art of infrared gas sensor self-calibration is the well-established ABC technology (Automatic Baseline Correction) where the sensor is calibrated to a fixed value that is assumed to be the fresh air gas concentration. However, this method has proven not to provide a sufficiently high accuracy when a high accuracy is requested. Thus, designing more robust and smart self-calibration algorithms, which can be widely applied in different environments, becomes more and more important.

### SUMMARY OF THE INVENTION

An object of the present invention is to provide a method for automatic calibration of measurement values from a spectroscopic sensing unit, such as an, e.g., non-dispersive infrared, NDIR, sensing unit, which provides a more reliable calibration than the methods according to the prior art. Another object of the present invention is to provide a method for automatic calibration of measurement values from a spectroscopic sensing unit, which takes care of aging of spectroscopic sensing units in a better way than the methods according to the prior art.

Another object of the present invention is to provide a gas sensor device comprising a spectroscopic sensing unit such as an, e.g., non-dispersive infrared, NDIR, sensing unit, which provides a more reliable calibration than the methods according to the prior art.

Another object of the present invention is to provide a gas sensor device comprising a spectroscopic sensing unit such as an, e.g., non-dispersive infrared, NDIR, sensing unit, which takes care of aging of sensing units in a better way than the methods according to the prior art.

Another object of the present invention is to provide a computer program for automatic calibration of measurement values from a spectroscopic sensing unit such as an, e.g., non-dispersive infrared, NDIR, gas sensing unit, which provides a more reliable calibration than the methods according to the prior art.

Another object of the present invention is to provide a computer program for automatic calibration of measurement values from a spectroscopic sensing unit such as an, e.g., non-dispersive infrared, NDIR, gas sensing unit, which takes care of aging of spectroscopic gas sensing units in a better way than the methods according to the prior art.

At least one of these objects is fulfilled with a method and a computer program according to the independent claims.

Further advantages are achieved with the features of the dependent claims.

According to a first aspect of the invention a gas sensor device is provided which comprises a spectroscopic sensing unit, a memory and a control unit, wherein the control unit is configured to output calibrated values, which are measures of a gas concentration measured by the spectroscopic sensing unit, wherein the calibrated values are determined from measurement values obtained from the spectroscopic sensing unit and a baseline calibration parameter retrieved from the memory. The gas sensor device is characterized in that the control unit is configured to update the baseline calibration parameter by identifying the minimum measurement value obtained during a predetermined first time period, obtaining a time for the first time period, obtaining a model value for the obtained time, determining an updated baseline calibration parameter based on the minimum measurement value and the model value, and updating the baseline calibration parameter stored in the memory.

The measurement values may be electrical signals such as currents from an intensity sensor (not shown) which intensity sensor measures the intensity of light that has been transmitted through the gas component to be measured.

The measurement values may correspond to intensity signals. The measurement values may have a non-linear relationship with the light intensity.

The measurement values may be converted in the control unit to calibrated values, which are measures of a gas concentration measured by the spectroscopic sensing unit. The calibrated values may be concentration values of the gas component to be measured.

The calibrated values may be determined as a function of the measurement values and the baseline calibration parameter. By setting the function of the measurement value to be equal to the model value and using the minimum measurement value the equation may be solved to determine an updated baseline calibration parameter.

In gas sensor devices according to the prior art the baseline calibration parameter has been a fixed value. The calibration of the gas sensor devices according to the prior art have usually been performed with predetermined intervals, such as a predetermined number of times per year. In case the gas component to be measured is CO₂, it has been assumed that the carbon dioxide concentration varies due to different human activities such as traffic with cars having internal combustion engines and industrial activities such as fossil fuel power plants. It has also been assumed that the carbon dioxide concentration sometimes reach the background level such as when the traffic is at a minimum and the power plants produce no power and/or when a strong wind is blowing. It has been assumed in the prior art gas sensors that the background carbon dioxide concentration has a fixed value of, e.g., 400 ppm. At every calibration occasion the gas sensor device according to the prior art has retrieved the lowest measurement value during a preceding time period such as, e.g., the previous week and adjusted the baseline calibration parameter so that the gas sensor device outputs a correct carbon dioxide concentration. With the gas sensor device according to this application the baseline calibration parameter follows a predicted curve.

The gas sensor device may be configured such that the calibrated values corresponds to concentrations of a gas component such as, e.g., carbon dioxide. The calibrated values may be determined as a function of the measurement values and the baseline calibration parameter. The model value corresponds to a model gas concentration and may be obtained in different ways as described below. The conversion function may incorporate the Beer-Lambert law, and may also take into account environmental factors. The conversion function can be described as follows

$$M = f ( zero , E , F ) ,$$

where M is the carbon dioxide concentration, E denotes the measurement value from the spectroscopic sensing unit, F denotes environmental factors, and zero denotes a baseline calibration parameter. The environmental factors may take into account factors such as the ambient temperature, the atmospheric pressure and the humidity and may be fixed or omitted in case the ambient temperature, the atmospheric pressure and the humidity are constant.

The control unit may be configured to determine the carbon dioxide concentration M using the conversion function.

$$M = f ( zero , E , F ) .$$

The conversion function may be an exponential function due to the Beer-Lambert relation on which the measurement value is dependent.

The control unit may be configured to calculate an updated baseline calibration parameter by setting M equal to the model value E equal to the minimum measurement value. The environmental factors F are set according to separate measurements of said factors. To obtain an updated baseline calibration parameter the equation is then solved. There are many different ways of solving such an equation known from the prior art sensor devices. However, in the prior art sensor devices it was assumed that the background carbon dioxide concentration had a fixed value of, e.g., 400 ppm.

A set of model values for different times may be stored in the memory together with their associated times. The model value may be obtained by retrieving from the memory the model value associated with the obtained time. The model values have been calculated in advance using a mathematical model for a plurality of different times. The mathematical model will be described in more detail below. The number of different model values should be adapted to the desired calibration interval and to the expected lifetime of the gas sensor device.

As an alternative to having a plurality of model values pre-stored in the memory of the gas sensor device the model values may be obtained by retrieving a set of model coefficients from the memory, calculating a model value, with a mathematical model being a function of time, using the obtained time and using the retrieved model coefficients in the mathematical model. If storage space in the memory is limited, this may be preferable.

The mathematical model may be hard wired in the control unit. This would provide a shorter time for calculating the model values.

The mathematical model may be a quadratic polynomial with a periodic term. Such a polynomial would enable a good fit to the historical CO₂ values that have been measured at different geographical positions if CO₂ is the gas component to be measured. The periodic term may comprise a sinus term and may comprise a sinus term within the sinus function to take into account that the CO₂ level is falling more rapidly during the summer than it is rising in the winter.

A plurality of sets of coefficients may be stored in the memory, wherein each set of coefficients is related to a geographical position, and wherein the control unit retrieves a set of coefficients, to be used for calculating the model measurement value, based on information on the geographical position of the gas sensor device. In this way, the mathematical model may be adapted to different geographical positions. As an example, the coefficients should be different when the gas sensor device is located with large forests surrounding it, than when the gas sensor device is located at a small island in the ocean. Another important factor is whether the gas sensor is located on the southern hemisphere or the northern hemisphere. The geographical position of the gas sensor device may be set by an operator.

When pre-calculated model values are stored in the gas sensor device a plurality of sets of model values for different times may be stored in the memory together with their associated times, wherein each set of model values is related to a geographical position, and wherein the control unit retrieves a model value based also on information on the geographical position of the gas sensor device. The geographical position of the gas sensor device may be set by an operator.

The gas sensor device may comprise a positioning device configured to determine the geographical position of the gas sensor device, wherein the control unit is configured to retrieve a geographical position from the positioning device and to retrieve the set of calibration coefficients corresponding to the retrieved position. The positioning device may be a satellite-positioning device such as, e.g., a GPS positioning device. By comprising a positioning device, the gas sensor device may automatically determine its own position.

The gas sensor device may comprise an internal clock. In this way, the time for the above-described first time period may be determined. The gas sensor device may alternatively be configured to obtain the time for the above-described first time period from an external clock. The external clock may be of many different sorts. If the gas sensor device comprises a positioning device such as a GPS positioning device, time may be obtained from the positioning device. As another alternative, the external clock may be a clock device that transmits the time by radio signals. The clock in such a clock device may be an atomic clock. The time may also be obtained from a cellular network. In cellular networks, a time is transmitted from base stations. The above are only a few examples on external clocks from which the time may be obtained.

It is not necessary to have model coefficients of model values stored in the memory. As an alternative, the control unit is configured to retrieve from the memory the measurement values from a predetermined second time period, and to set the calibration coefficients so that the mathematical model fits the measurement values. In this way, the control unit presumes that the measurements that have been made during the second time period after installation at a location are correct. The coefficients of the mathematical model is set to fit the measurements values. The mathematical model of course have to be stored in the gas sensor device.

Above, the model values have been described as values. However, each model value may be associated with an uncertainty.

The uncertainty associated with each model value may be an uncertainty function, which is based on an earlier set of measurement values. In other words, a set of measurement values may be used to determine the uncertainty function for a model value.

The uncertainty may alternatively be expressed as the standard deviation from the model value or any other statistical measure that can be used to describe the uncertainty.

The uncertainty function may be based on measurement values measured by the sensor device itself and/or by at least one other gas sensor device. The measurement values may have been obtained during one or several previous years.

According to a second aspect of the present invention a computer implemented method is provided for updating the baseline calibration parameter stored in a memory and used to determine calibrated values, which are measures of a gas concentration measured by an spectroscopic sensing unit, wherein the calibrated values are determined from measurement values obtained from the spectroscopic sensing unit and the baseline calibration parameter. The computer implemented method is characterized in that the method comprises the steps of obtaining measurement values from the spectroscopic sensing unit, identifying the minimum measurement value obtained during a predetermined first time period, obtaining a time for the first time period, obtaining a model value for the obtained time, determining an updated baseline calibration parameter based on the minimum measurement value and the model value, and updating the baseline calibration parameter stored in the memory. The method according to the second aspect may be performed in a so-called computer cloud.

The model values may be obtained by retrieving a set of model coefficients from the memory, calculating a model value, with a mathematical model being a function of time, using the obtained time and using the retrieved model coefficients in the mathematical model.

A plurality of sets of coefficients may be stored in the memory, wherein each set of coefficients is related to a geographical position. The method may comprise the steps of obtaining information on the geographical position related to the measurement values, and retrieving a set of coefficients, to be used for calculating the model measurement value, based also on the obtained information on the geographical position related to the measurement values. The information on the geographical position related to the measurement values may be obtained from the gas sensor device or from the memory. In the latter case, the measurement values have to be related to an identification code identifying the gas sensor device. The geographical position may be stored in a look up table together with the identification code.

A set of model values for different times may be stored in the memory together with their associated times, and wherein the model value is obtained by retrieving the model value associated with the obtained time from the memory. The model values have been calculated in advance using a mathematical model for a plurality of different times. The mathematical model will be described in more detail below. The number of different model values should be adapted to the desired calibration interval and to the expected lifetime of the gas sensor device.

A plurality of sets of model values for different times may be stored in the memory together with their associated times, wherein each set of model values is related to a geographical position. The method may comprise the steps of obtaining information on the geographical position related to the measurement values, and retrieving a model value, to be used for calculating the model measurement value, based also on the obtained information on the geographical position related to the measurement values. The information on the geographical position related to the measurement values may be obtained from the gas sensor device or from the memory. In the latter case, the measurement values have to be related to an identification code identifying the gas sensor device. The geographical position may be stored in a look up table together with the identification code.

As described above for the gas sensor device according to the first aspect of the present invention each model value may be associated with an uncertainty.

The uncertainty associated with each model value may be an uncertainty function, which is based on an earlier set of measurement values. In other words, a set of measurement values may be used to determine the uncertainty function for a model value.

The uncertainty may alternatively be expressed as the standard deviation from the model value or any other statistical measure that can be used to describe the uncertainty.

The uncertainty function may be based on measurement values measured by the sensor device itself and/or by at least one other gas sensor device. The measurement values may have been obtained during one or several previous years.

According to a third aspect of the present invention a computer program for updating the baseline calibration parameter stored in a memory and used to determine calibrated values, which are measures of a gas concentration measured by an spectroscopic sensing unit, wherein the calibrated values are determined from measurement values obtained from the spectroscopic sensing unit and the baseline calibration parameter, comprising instructions which, when executed by a processor in a processing unit causes the processing unit to control the processing unit to carry out the method according to the second aspect.

The spectroscopic sensing unit may be a non-dispersive infrared, NDIR, sensing unit, which is a commonly used type of spectroscopic sensing unit.

### BRIEF DESCRIPTION OF THE DRAWINGS

FIG. 1 shows schematically a gas sensor device.

FIG. 2 shows the monthly mean CO₂ concentration at Mauna Loa from 1958 to 2020.

FIG. 3 shows as a solid line the CO2 concentration on a geographical position on the northern hemisphere together with a mathematical model of the CO₂ concentration as a dashed line.

FIG. 4 shows a comparison between curves obtained with a high precision gas sensor, a low precision gas sensor and the low precision gas sensor calibrated with the method described in this application.

FIG. 5 shows curves obtained with a high precision gas sensor, a low precision gas sensor after calibration with the method according to the prior art and with a low precision gas sensor after calibration with the method described in this application.

FIG. 6 shows a gas sensor device 1 in communication with a remote device 20 and illustrates a method according to a different embodiment.

FIG. 7 shows nine different clusters of measurement curves measured obtained during about two years of measurements.

FIG. 8 shows the nine different clusters of FIG. 7 combined.

### DETAILED DESCRIPTION

In the following detailed description of the invention, similar features in the different figures will be denoted with the same reference numeral.

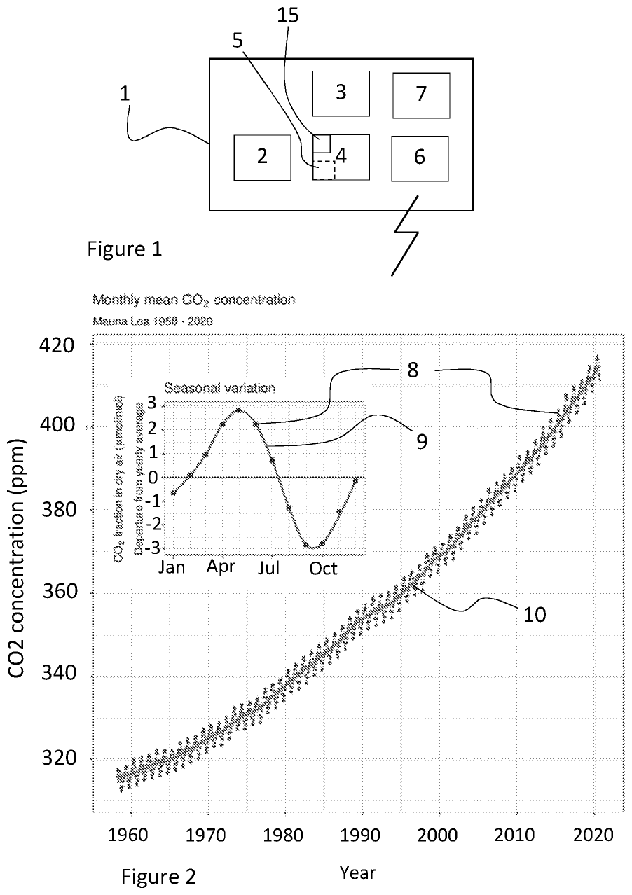

FIG. 1 shows schematically a gas sensor device 1 comprising a spectroscopic sensing unit 2 such as, e.g., a non-dispersive infrared, NDIR, sensing unit, a memory 3 and a control unit 4 with a processor 5, wherein the control unit 4 obtains measurement values from the spectroscopic sensing unit 2. The measurement values from the spectroscopic sensing unit are dependent on the concentration of a gas component, which the spectroscopic sensing unit 2 is configured to measure. The spectroscopic sensing unit 2 measures at an absorption peak of the gas component and the measurement value depends on the gas concentration according to the Beer-Lambert law. The measurement signal may be proportional to a detected light intensity. The function of spectroscopic sensing units 2 is well known from the prior art and will not be explained further herein.

The control unit 4 is configured to output calibrated values, which are measures of a concentration of a gas component measured by the spectroscopic sensing unit 2. The gas sensor device is primarily intended for measurements of the carbon dioxide concentration in the atmosphere. To be able to output the calibrated values the gas sensor device 1 comprises a communication interface 6, which is configured to communicate wirelessly with a remote communication device (not shown) such as a base station (not shown) or any other form of transmitter or transceiver. As an alternative, the communication device may be configured for communication by wire. The calibrated values are determined from measurement values obtained from the spectroscopic sensing unit 2 and a baseline calibration parameter retrieved from the memory. Also shown in FIG. 1 is an optional internal clock 15 in the control unit and an optional positioning device 7.

The measurement values may be intensity values of the light that penetrates the gas to be measured. The spectroscopic sensing unit 2 is preferably configured to measure the light intensity in a specific wavelength interval. The gas sensor may also be configured to transform the intensity value to a gas concentration. In this case, the measure of the gas concentration is the gas concentration. If the transformation from the light intensity to the gas concentration is known to the processing unit performing the method, it is possible to use the light intensity. If, however, the transformation from the light intensity to the gas concentration is not known to the processing unit the belief functions are preferably a probability as a function of the gas concentration.

In gas sensor devices according to the prior art the baseline calibration parameter has been a fixed value. The calibration of the gas sensor devices according to the prior art have usually been performed with predetermined intervals, such as a predetermined number of times per year. It has been assumed that the carbon dioxide concentration varies due to different human activities such as traffic with cars having internal combustion engines and industrial activities such as fossil fuel power plants. It has also been assumed that the carbon dioxide concentration sometimes reach the background level such as when the traffic is at a minimum and the power plants produce no power and/or when a strong wind is blowing. It has been assumed in the prior art gas sensors that the background carbon dioxide concentration has a fixed value of, e.g., 400 ppm. At every calibration occasion the gas sensor device according to the prior art has retrieved the lowest measurement value during a preceding time period such as, e.g., the previous week and adjusted the calibration parameter so that the gas sensor device outputs a correct carbon dioxide concentration. In the described method according to the prior art the minimum measurement value is converted to a carbon dioxide concentration using a conversion function which can be described as follows

$$M = f ( zero , E , F ) ,$$

where M is the carbon dioxide concentration, E denotes the measurement value from the spectroscopic sensing unit 2, F denotes an environmental factor, and zero denotes a baseline calibration parameter. The baseline calibration parameter has to be adjusted over time due to aging of the gas sensing unit 2. This has been done in the prior art by assuming that a lowest carbon dioxide concentration during a fixed time period is 400 ppm. The time period has typically been chosen to be one week.

The inventors have realised that this approximation is not satisfactory if high accuracy in the concentration measurement is desired or if the sensor is to be used for many years. The reason for this is that the carbon dioxide concentration in the atmosphere varies over the year and increases from year to year. FIG. 2 shows the monthly mean CO₂ concentration at Mauna Loa from 1958 to 2020 as dots 8. The solid line 9 in FIG. 2 is the trend of the CO₂ concentration. The inset in FIG. 2 shows in an enlargement the seasonal variation of the mean CO₂ concentration as the departure from the yearly average with the solid line 10 being a fit to the monthly averages. The curve 10 is known as the Keeling curve. As can be observed the overall CO₂ concentration is increasing with cyclical fluctuations of about ±3 ppm. The reason for the cyclical fluctuations is the seasons on the Northern hemisphere. During summer, the vegetation absorbs more CO₂, which results in a decrease in the concentration of CO₂ in the atmosphere. On the Southern hemisphere, the summer is phase shifted by about 6 months and the decrease of the CO₂ concentration is in a corresponding way phase shifted 6 months. Due to the trade winds, the mixing of the air in the atmosphere is limited across the equator.

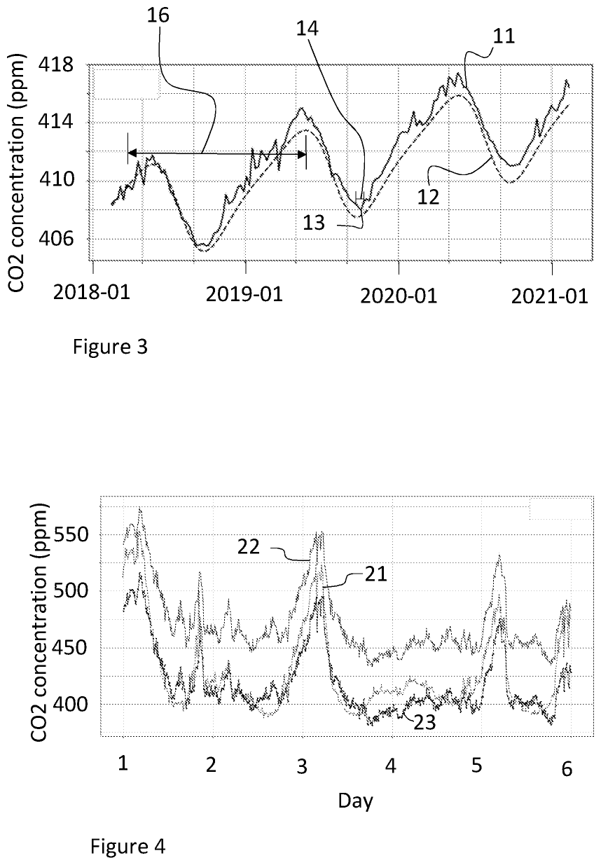

FIG. 3 shows as a solid line 11 the CO2 concentration of the latest 3 years on a geographical position on the northern hemisphere together with a mathematical model of the CO₂ concentration as a dashed line 12. As can be seen in FIG. 3 the cyclical decrease is more rapid that the cyclical increase of the CO₂ concentration in the atmosphere. The concentration values in FIG. 3 have been obtained by converting the measurement values to a concentration according to a known conversion function as described above:

$$M = f ( zero , E , F ) ,$$

The mathematical model shown as the dotted line 12 is a quadratic polynomial with a periodic term. In the present example, the periodic term is a sinus term. The model used for the concentration shown in FIG. 3 is

$$y = {c}_{0} + {c}_{1} x + {c}_{2} {x}^{2} + ( {c}_{3} + {c}_{4} x ) sin ( {c}_{5} sin ( k x + {c}_{6} ) + k x + {c}_{7} ) ;$$

where y is the concentration of CO₂ and x is the time in days. In the model of FIG. 3, the following values have been used for c₀-c₇:

[3.145×10²,2.056×10⁻³,9.939×10⁻⁸,2.852,2,495×10⁻⁵,5.024×10⁻¹,9×10⁻¹,1.145].

It is of course possible to use a simpler model if a lower accuracy is acceptable. Such a simpler model may be achieved by simply setting one or more of the coefficients c₀-c₇ to zero. It is preferable that the mathematical model is a quadratic polynomial with a periodic term as this reflects the increase of the CO₂ concentration in the atmosphere. The period term should have a periodicity of 1 year. That means that the term k should be equal to 2π/365.25. The term c₇ is a phase shift that is different on the northern hemisphere and the southern hemisphere.

The control unit 4 is configured to update the baseline calibration parameter by identifying the minimum measurement value 13 obtained during a predetermined first time period 14 shown in FIG. 3. This can be done either by continuously storing the minimum measurement value or by storing all measurement values and then identifying the minimum. In the example of FIG. 3, it is the minimum measurement value 12 after conversion to a concentration that is identified. The conversion is made using the function

$$M = f ( zero , E , F ) .$$

It would also be possible to identify the minimum measurement value 12 before conversion and then convert the minimum measurement value 12. Usually, the predetermined first time period 14 is on the order of 1 week, but in FIG. 3 the first time period is about a month. The control unit obtains a time for the first time period. As can be seen in FIG. 3 the variation even within a month is small. Thus, it is not necessary to have the exact time for the minimum measurement value 13 as the time for the first time period 14. The time for the first time period can be the time for the minimum measurement value or an arbitrary time between the beginning and the end of the first time period 14. The control unit may retrieve a model value from the memory 3 for the determined time. Model values for several years may be stored in the memory 3 together with their corresponding time. If the baseline calibration parameter is updated only once a week, the necessary number of baseline calibration values is only fifty-two for each year. As an alternative, the control unit 4 may retrieve a set of model coefficients from the memory 3 and calculate a model value, with a mathematical model being a function of time, using the obtained time for the first time period 14 and using the retrieved model coefficients in the mathematical model. The mathematical model used is as described above and may either be hardwired in the control unit 4 or may be retrieved from the memory 3. Irrespective of how the model value is obtained, the control unit 4 then determines an updated baseline calibration parameter based on the minimum measurement value and the model value, and updates the baseline calibration parameter stored in the memory 3. In the example shown in FIG. 3 the measurement values have been converted into CO₂ concentrations using the function M and the present calibration parameter. The minimum in the first time period is above the model. This would result in that an updated calibration parameter is determined which results in lower CO₂ concentrations.

The gas sensor device 1 may comprise an internal clock 5, which provides the necessary time for the measurement values. Alternatively, the gas sensor device 1 may obtain the time from an external clock using the communication interface 6. In this case, the internal clock 5 may be omitted. The external clock may be of many different sorts. If the gas sensor device comprises a positioning device such as a GPS positioning device, time may be obtained from the positioning device. As another alternative, the external clock may be a clock device that transmits the time by radio signals. The clock in such a clock device may be an atomic clock. The time may also be obtained from a cellular network. In cellular networks, a time is transmitted from base stations. The above are only a few examples on an external clock from which a time may be obtained.

In case all measurement values are stored in the memory 3, they are stored with the corresponding time for each measurement value. In case only the minimum measurement value is stored in the memory, the corresponding time is also stored in the memory 3.

The gas sensor device 1 may be configured with a plurality of sets of model values for different times stored in the memory together with their associated times. Alternatively, the gas sensor device 1 may be configured with a plurality of sets of coefficients stored in the memory 3, wherein each set of coefficients is related to a geographical position. As described above the Keeling curve is different at different geographical positions. As mentioned above the cyclic variations due to seasons are phase shifted by about 6 months on the southern hemisphere in comparison with the northern hemisphere. The control unit may retrieve model values from one of the sets of model values, wherein the choice of set of model values is based on information on the geographical position of the gas sensor device. Correspondingly, for the case with a plurality of sets of coefficients stored in the memory 3, the control unit retrieves a set of coefficients, to be used for calculating the model measurement value, based on information on the geographical position of the gas sensor device 1. The position of the gas sensor device 1 may be input by an operator, which arranges the gas sensor device at a location where it is to measure the CO₂ concentration. Alternatively, the gas sensor device 1 comprises a positioning device 7 configured to determine the geographical position of the gas sensor device, wherein the control unit is configured to retrieve a geographical position from the positioning device 7 and to retrieve the set of calibration coefficients corresponding to the retrieved position. The positioning device 7 may use a satellite positioning system such as GPS or GLONASS. By having such a positioning device, the control unit may obtain the position of the gas sensor device from the positioning device 7. With the obtained position, the control unit may retrieve the correct set of model coefficients from the memory 3. When the gas sensor device 1 comprises a positioning device 7 the control unit 4 may retrieve the time from the positioning device 7 as most satellite positioning systems are based on a very accurate clock.

The control unit 4 may additionally or alternatively be configured to retrieve from the memory the measurement values from a predetermined second time period 16 as shown in FIG. 3, which is longer than the first time period and preferably at least a year. The control unit determines the set of model coefficients so that the mathematical model fits the measurement values in the second time period 16. The determined set of model coefficient is then used in later calibrations of the sensor device 1.

FIG. 4 shows a first dashed curve 21 which has been obtained with a high precision gas sensor, a second solid curve 22 which has been obtained with a low precision gas sensor and a third dotted curve 23 which is the second curve 22 calibrated with the method described above.

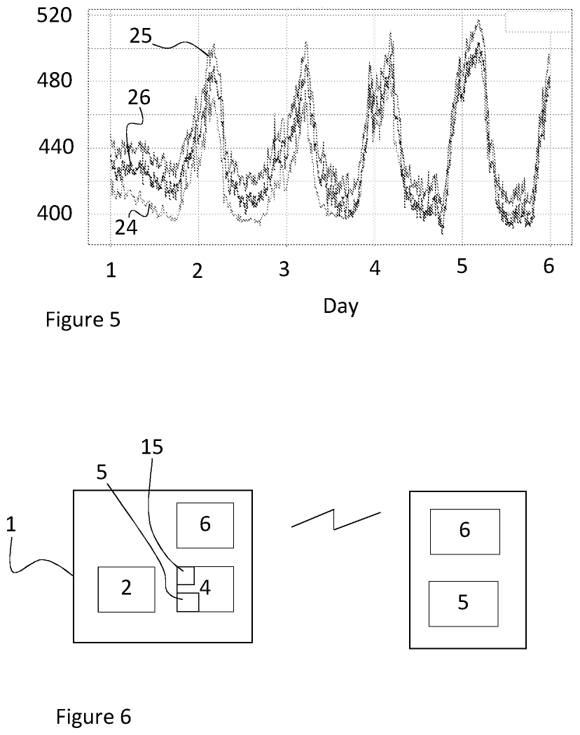

FIG. 5 shows a first dashed curve 24 which has been obtained with a high precision gas sensor, a second solid curve 25 obtained with a low precision gas sensor after calibration with the method according to the prior art with a fixed baseline calibration parameter and a third dotted curve 26 obtained with a low precision gas sensor after calibration with the method according to the present invention.

It is not necessary to have the gas sensor device configured to do the conversion of the measurements values to gas concentration values with the use of measurement values and a baseline calibration parameter, and to update baseline calibration parameter in the gas sensor device. As an alternative the gas sensor device may send all measurement values to a remote computer, which may be a virtual computer, usually called a cloud computer.

FIG. 6 shows a gas sensor device 1 in communication with a remote device 20 and illustrates a method according to a different embodiment. The gas sensor device 1 comprises a non-dispersive infrared, spectroscopic, sensing unit 2, a memory 3 and a control unit 4 with a processor 5 and an internal clock, wherein the control unit 4 is configured to transmit measurement values obtained with the spectroscopic sensing unit 2, which are dependent on the concentration of a component in gas sensed by the spectroscopic sensing unit 2. The measurement values are transmitted together with their corresponding time. The gas sensor device is primarily intended for measurements of the carbon dioxide concentration in the atmosphere. To be able to output the calibrated values the gas sensor device 1 comprises a communication interface 6, which is configured to communicate wirelessly with a remote communication device 6′ which is arranged in a remote device 20. The communication interface 6′ of the remote device 20 receives the measurement values and their corresponding times from the communication interface 6 of the gas sensor device. The processor 5′ of the remote device is in communication with a memory 3. The processor then performs the method as has been described above. In case the geographical position of the gas sensor device 1 is required, the remote device may receive the geographical position from the gas sensor device 1. Alternatively, the remote device may receive an identification number from the gas sensor device 1. The remote device may then retrieve the position of the gas sensor device 1 from a database by using the identification number.

The measurement values may be transmitted either one by one or in groups with a plurality of measurement values.

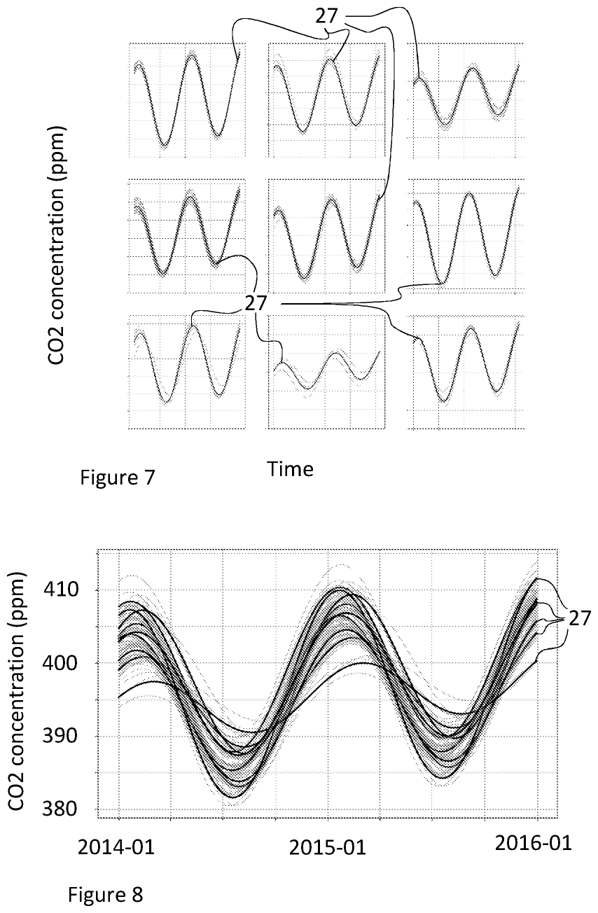

FIG. 7 shows nine different clusters of measurement curves measured obtained during about two years of measurements. Each cluster comprises a plurality of measurement curves obtained during two years of measurements with different sensors positioned in the same geographical area such as, e.g., northern Sweden. All clusters have been obtained in the same larger geographical area such as, e.g., Europe. The measurement curves in each one of the clusters have a spread. The spread may be used to determine an uncertainty in the model values. The uncertainty may be expressed as a standard deviation from the model value such as, e.g., 400 ppm±10 ppm. Alternatively, the uncertainty may be expressed as a probability function for each model value. The mean curve in each cluster is shown as a thick line 27.

FIG. 8 shows the nine different clusters of FIG. 7 combined. If a model value is to be used for the larger geographical area represented by all nine clusters the uncertainty will be larger as is illustrated by the larger spread of the curves in FIG. 8. The mean curves from each cluster is shown as a thick line 27.

The above-described embodiments may be amended in many ways without departing from the scope of the invention, which is limited only by the appended claims.

1. A gas sensor device comprising aft spectroscopic sensing unit, a memory and a control unit, wherein the control unit is configured to output calibrated values, which are measures of a concentration of a gas component measured by the spectroscopic sensing unit, wherein the calibrated values are determined from measurement values obtained from the spectroscopic sensing unit and a baseline calibration parameter retrieved from the memory,
wherein the control unit is configured to update the baseline calibration parameter (zero) by
identifying the minimum measurement value obtained during a predetermined first time period,
obtaining a time for the first time period,
obtaining a model value corresponding to the obtained time,
determining an updated baseline calibration parameter based on the minimum measurement value and the model value, and
updating the baseline calibration parameter stored in the memory.

2. The gas sensor device according to claim 1, wherein the calibrated values corresponds to gas concentrations and are determined as a function of the measurement values and the baseline calibration parameter, and wherein the model value corresponds to a model gas concentration.

3. The gas sensor device according to claim 1, wherein a set of model values for different times are stored in the memory together with their associated times, and wherein the model value is obtained by retrieving from the memory the model value associated with the obtained time.

4. The gas sensor according to claim 1, wherein the model values are obtained by
retrieving a set of model coefficients from the memory,
calculating a model value, with a mathematical model being a function of time, using the obtained time and using the retrieved model coefficients in the mathematical model.

5. The gas sensor device according to claim 4, wherein the mathematical model is hard wired in the control unit.

6. The gas sensor device according to claim 5, wherein the mathematical model is a quadratic polynomial with a periodic term.

7. The gas sensor device according to claim 6, wherein a plurality of sets of coefficients are stored in the memory, wherein each set of coefficients is related to a geographical position, and wherein the control unit retrieves a set of coefficients, to be used for calculating the model measurement value, based on information on the geographical position of the gas sensor device.

8. The gas sensor device according to claim 3, wherein a plurality of sets of model values for different times are stored in the memory together with their associated times, wherein each set of model values is related to a geographical position, and wherein the control unit retrieves a model value based also on information on the geographical position of the gas sensor device.

9. The gas sensor device according to claim 8, comprising a positioning device configured to determine the geographical position of the gas sensor device, wherein the control unit is configured to retrieve a geographical position from the positioning device and to retrieve the set of calibration coefficients corresponding to the retrieved position.

10. The gas sensor device according to claim 9, comprising an internal clock.

11. The gas sensor device according to claim 10, wherein the control unit is configured to retrieve from the memory the measurement values from a predetermined second time period, and to set the calibration coefficients so that the mathematical model fits the measurement values.

12. The gas sensor device according to claim 1, wherein each model value is associated with an uncertainty.

13. A computer implemented method for updating the baseline calibration parameter stored in a memory and used to determine calibrated values, which are measures of a concentration of a gas component measured by an spectroscopic sensing unit, wherein the calibrated values are determined from measurement values obtained from the spectroscopic sensing unit and the baseline calibration parameter,
characterized in that the method comprises the steps of
obtaining measurement values from the spectroscopic sensing unit,
identifying the minimum measurement value obtained during a predetermined first time period,
obtaining a time for the first time period,
obtaining a model value for the obtained time,
determining an updated baseline calibration parameter based on the minimum measurement value and the model value, and
updating the baseline calibration parameter stored in the memory.

14. The computer implemented method according to claim 13, wherein the model values are obtained by
retrieving a set of model coefficients from the memory,
calculating a model value, with a mathematical model being a function of time, using the obtained time and using the retrieved model coefficients in the mathematical model.

15. The computer implemented method according to claim 14, wherein a plurality of sets of coefficients are stored in the memory, wherein each set of coefficients is related to a geographical position, comprising the steps of
obtaining information on the geographical position related to the measurement values, and
retrieving a set of coefficients, to be used for calculating the model measurement value, based also on the obtained information on the geographical position related to the measurement values.

16. The computer implemented method according to claim 13, wherein a set of model values for different times are stored in the memory together with their associated times, and wherein the model value is obtained by retrieving, from the memory, the model value that is associated with the obtained time.

17. The computer implemented method according to claim 16, wherein a plurality of sets of model values for different times are stored in the memory together with their associated times, wherein each set of model values is related to a geographical position, comprising the steps of
obtaining information on the geographical position related to the measurement values, and
retrieving a model value, to be used for calculating the model measurement value, based also on the obtained information on the geographical position related to the measurement values.

18. The computer implemented method according to claim 17, wherein each model value is associated with an uncertainty.

19. A non-transitory storage medium comprising a computer program for updating the baseline calibration parameter stored in a memory and used to determine calibrated values, which are measures of a gas concentration measured by an spectroscopic sensing unit, wherein the calibrated values are determined from measurement values obtained from the spectroscopic sensing unit and the baseline calibration parameter, comprising instructions which, when executed by a processor in a processing unit causes the processing unit to control the processing unit to carry out the method according to claim 18.
